(19) United States
(12) Patent Application Publication
(10) Pub. No.: US 2015/0224188 A1
(43) Pub. Date: Aug. 13, 2015

(54) USE OF MEMBRANE VESICLE-BASED VACCINE AGAINST M. TUBERCULOSIS

(71) Applicant: ALBERT EINSTEIN COLLEGE OF MEDICINE OF YESHIVA UNIVERSITY, Bronx, NY (US)

(72) Inventors: Arturo Casadevall, New York, NY (US); Rafael Prados-Rosales, New York, NY (US); Steven A. Porcelli, Hartsdale, NY (US); William R. Jacobs, Pelham, NY (US)

(73) Assignee: ALBERT EINSTEIN COLLEGE OF MEDICINE OF YESHIVA UNIVERSITY, Bronx, NY (US)

(21) Appl. No.: 14/429,001

(22) PCT Filed: Sep. 23, 2013
(86) PCT No.: PCT/US13/61120

§ 371 Date: Mar. 18, 2015

Related U.S. Application Data
(60) Provisional application No. 61/704,874, filed on Sep. 24, 2012.

(51) Int. Cl. A61K 39/39; A61K 39/04

(52) U.S. Cl. CPC A61K 39/39; A61K 39/04; A61K 2039/55555

(57) ABSTRACT

Provided are compositions comprising a plurality of isolated mycobacterium membrane vesicles, and methods of use thereof, and methods of improving the efficacy of immunizations. Throughout this application various publications are referred to by number in parentheses. Full citations for the references may be found at the end of the specification. The disclosures of each of these publications, and also the disclosures of all patents, patent application publications and books recited herein, are hereby incorporated by reference in their entirety into the subject application to more fully describe the art to which the subject invention pertains.

### CROSS-REFERENCE TO RELATED APPLICATIONS

This application claims benefit of U.S. Provisional Application No. 61/704,874, filed Sep. 24, 2012, the contents of which are hereby incorporated by reference.

### BACKGROUND OF THE INVENTION

Throughout this application various publications are referred to by number in parentheses. Full citations for the references may be found at the end of the specification. The disclosures of each of these publications, and also the disclosures of all patents, patent application publications and books recited herein, are hereby incorporated by reference in their entirety into the subject application to more fully describe the art to which the subject invention pertains.

Mycobacterium tuberculosis, the causative agent of Tuberculosis (TB), remains a leading human pathogen and tuberculosis is a major health problem in the developing world. In 2010 there were 8.8 million incident cases of TB, 1.1 million deaths from TB among HIV-negative people and an additional 0.35 million deaths from HIV-associated TB (1). Efforts to control the disease include the development of “point-of-care” tests, new TB drugs, the use of the Bacillus Calmette-Guerin (BCG) vaccine and the development of new vaccines. Most of the new vaccine candidates against TB that have entered in clinical trials fall into one of the following groups: (I) live attenuated vaccines to replace BCG; (II) subunit vaccines to be given on after initial BCG vaccination (2); and (III) single immunodominant antigens, usually secreted, such as ESAT-6, Cfp10 and Ag85b (2).

Bacterial pathogens have developed different secretion systems to release their products to the extracellular environment, tissues or bloodstream of the host organism (3). Some microorganisms including bacteria and fungi use membrane vesicles (MVs), to release a complex group of proteins, polysaccharides and lipids into the extracellular milieu (3-7). Both pathogenic and nonpathogenic species bacteria secrete vesicles consistent with the notion that they are means by which bacteria interact with prokaryotic and eukaryotic cells in their environment (3). Typically, vesicles from pathogenic bacteria contain virulence factors including toxins, adhesins or immunomodulatory compounds. The packaging of virulence factors in vesicles allows microorganisms to deliver host cell damaging materials in a concentrated manner. This laboratory has recently demonstrated the production of MVs in many mycobacterium species, including the medically important BCG and Mtb (7). It was shown that mycobacterial MVs transport lipids and proteins previously known to be involved in the subversion of the immune response of the host. In addition, it has been demonstrated that these vesicles trigger an inflammatory response in a TLR2 dependent manner that directly modulates the outcome of the interaction with the host, contributing to virulence and pathogenesis in Mtb infection (7).

Despite the intimate connection between bacterial vesicles and virulence, they have also been used as a vaccine, eliciting immune responses that protect against mucosal and systemic bacterial infections such as Neisseria meningitidis (8), Bordetella pertussis (9), Salmonella typhimurium (10), Vibrio chloerae (11) and Bacillus anthracis (12). Artificially produced membrane vesicles from the gram-negative bacteria N. meningitidis constitute the basis of one the few licensed vesicle-based vaccines (VA-MENGOC-BCTM) (13). Recently, the immune response to artificial membrane vesicles from M. smegmatis was tested in mice, showing cross-reactivity against Mtb antigens (14).

The present invention address the need for improved vaccines and vaccination methods for tuberculosis.

### SUMMARY OF THE INVENTION

This invention provides a composition comprising a plurality of isolated mycobacterium membrane vesicles.

The invention also provides a method of eliciting an immune response in a subject comprising administering to the subject any of the vesicle compositions described herein an amount effective to elicit an immune response.

The invention also provides a method of improving the efficacy of an anti-mycobacterial immunization administered to a subject, comprising administering to the subject any of the compositions described herein in an amount effective to improve efficacy of an anti-mycobacterial immunization administered to a subject.

The invention also provides a method of immunizing a subject against tuberculosis comprising administering to the subject an amount of any of the instant compositions in an amount effective to immunize a subject against tuberculosis.

Also provided is a composition comprising a plurality of isolated bacterium membrane vesicles isolated from a pathogenic bacterium that produces membrane vesicles. In an embodiment, the composition further comprises an immunological adjuvant. In an embodiment, the composition further comprises a pharmaceutically acceptable carrier.

Also provided is a method of eliciting an immune response in a subject comprising administering to the subject any of the instant compositions in an amount effective to elicit an immune response.

Also provided is a method of preparing an isolated mycobacterium membrane vesicles preparation comprising centrifuging a culture of mycobacterium and treating the resultant product so as to remove live mycobacteria and recover the vesicles, thereby preparing the isolated mycobacterium membrane vesicles preparation.

Additional objects of the invention will be apparent from the description which follows.

### BRIEF DESCRIPTION OF THE DRAWINGS

FIG. 1A-1F. Immunogenicity of mycobacterial vesicles. (A) Titers of Mtb specific antibodies in serum from C57BL/6 mice (n=3 per group) immunized with 2.5 μg of BCG, H37Rv or M. smegmatis MVs using an intraperitoneal route of injection. (B) Immunoblot of serum reactivity for Mtb sonicate from two independently IP vaccinated mice. (C) Immunoblot using specific monoclonal antibodies for 19 kDa (lpqH) and LprG on Mtb sonicate. (D) Splenic IFN-γ producers T cells from MVs-immunized C57BL/6 mice (n=3 per group) after in vitro stimulation with Mtb sonicate or different subcellular fractions (E). Results are shown from one of three experiments. (F) Frequency of Mtb specific CD4+ T cells producing IFN-γ, TNF-α or IL-2 measured in splenocytes isolated from mice immunized with MVs via IP. Controls included sham (PBS) immunized mice and BCG Pasteur subcutaneously immunized mice for 4 weeks. The cytokine profile in individual cells was measured by multicolor flow cytometry by gating for CD4+ T cells. All possible combinations of cytokine expression were plotted. The not shown combinations mean that they were not detected. The results are representative from two independent experiments.

FIG. 2A-2D. Vaccine efficacy of mycobacterial MVs. (A, B) The bacterial load (CFUs) in the lungs (A) and spleens (B) of individual C57BL/6 mice, immunized with BCG MVs via IP or BCG Pasteur via SC mice, was determined at 4 weeks after infection with a low dose of Mtb via aerosol. The results are pooled values from two independent experiments. Experimental groups used from 5-8 mice. *P<0.05; **P<0.01 using one-way ANOVA. Data are means±s.e.m. (C) Representative H&E staining images from lungs of C57BL/6 mice immunized with BCG MVs via IP or BCG Pasteur via SC and challenged with Mtb. All the images are taken at magnification of 1.25×. (D) Quantification of histopathology by measuring the size of the lesions in lung on H&E images using ImageJ software (P<0.05 one-way ANOVA with Tukey post-test). (E,F) The bacterial load (CFUs) in the lungs (E) and spleens (F) of individual mice was determined at 9 weeks after infection with a low dose of Mtb via aerosol. The results are pooled values from two independent experiments. Mice groups ranged from 5-8. *P<0.05; **P<0.01, ***P<0.001 using one-way ANOVA. Data are means±s.e.m.

FIG. 3A-3E. Analysis of immunogenicity of MVs after boosting BCG. (A) Determination of specific antibodies to Mtb in serum from C57BL/6 mice (n=3 per group) immunized with 10⁶ BCG bacteria and boosted IP after two months with 2.5 μg of BCG, H37Rv or M. smegmatis MVs. (B) Immunoblot on Mtb sonicate using serum from two independent BCG vaccinated or IP MVs boosted-mice. (C) Specific splenic IFN-γ producers T cells from BCG vaccinated or IP MVs boosted-057BL/6 mice (n=3 per group) after in vitro stimulation with Mtb sonicate or different subcellular fractions (D). (E,F) Intracellular cytokine staining for IL-2, IFN and TNF of splenic CD4+ T cells from BCG vaccinated or IP vesicle-boosted mice (n=3 per group). All the possible combinations are shown. These results are representative of two independent experiments.

FIG. 4A-4C. Evaluation of the capacity of mycobacterial MVs to boost a BCG prime. (A,B) Bacterial numbers (CFUs) in lungs (A) and spleens (B) were determined 4 weeks after challenge with a low dose of Mtb via aerosol (n=6 per group) of C57BL/6 mice, previously vaccinated with BCG Pasteur for two months, or mice vaccinated with BCG and boosted with BCG MVs. Representative data from one of two experiments are shown as log 10 CFU (***P<0.001, one-way ANOVA with Tukey's post test). Data are means±s.e.m. (C) Representative H&E staining images from lung of C57BL/6 mice BCG vaccinated or BCG vaccinated and MVs boosted and aerosol infected with Mtb. All the images were taken at 1.25× magnification. (D) Quantification of histopathology of lung sections by measuring lesion size using ImageJ software (***P<0.001, one-way ANOVA with Tukey's post test). (E,F) The bacterial load (CFUs) in the lungs (E) and spleens (F) of individual mice was determined at 9 weeks after infection with a low dose of Mtb via aerosol. The results are pooled values from two independent experiments. Mice groups ranged from 5-8. *P<0.05; **P<0.01, ***P<0.001 using one-way ANOVA. Data are means±s.e.m.

FIG. 5A-5B. Enhanced humoral response toward mycobacterial membrane fraction. (A) Determination of specific antibodies to an Mtb membrane fraction in serum from C57BL/6 mice (n=3 per group) immunized with 2.5 μg of BCG, or H37Rv MVs using via IP or with BCG Pasteur via SC. (B) Immunoblot on Mtb membrane fraction using serum from two independent BCG or H37Rv MVs-vaccinated mice. Control included sera from two mice vaccinated with BCG Pasteur via SC for 4 weeks.

FIG. 6A-6C. Determination of specific antibodies to an Mtb membrane fraction in serum from C57BL/6 mice immunized with 2.5 μg M. smegmatis MVs using via IP.

FIG. 7A-7C. Determination of specific antibodies to an Mtb membrane fraction in serum from C57BL/6 mice subcutaneously immunized with 10⁶ BCG bacteria and boosted with 2.5 μg of BCG MVs via IP.

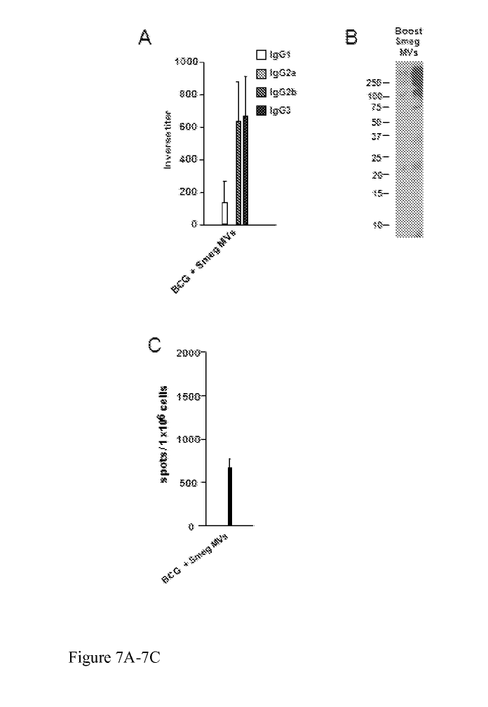

### DETAILED DESCRIPTION OF THE INVENTION

This invention provides a composition comprising a plurality of isolated mycobacterium membrane vesicles.

As used herein, “isolated” means not as existing in the natural state or not as existing in the natural environment. For example, a plurality of mycobacterium membrane vesicles recovered and suspended in a sterile carrier is not a natural state or natural environment. In an embodiment, the composition is synthetic in that it does not occur in nature.

In an embodiment, the composition further comprises an immunological adjuvant. In an embodiment, the composition further comprises a pharmacologically acceptable carrier. In an embodiment, the composition is sterile in not containing live bacteria.

In an embodiment, the composition comprises purified mycobacterium membrane vesicles.

In an embodiment, the composition is a vaccine for inducing an immune response against the mycobacterium in a mammal. In an embodiment, the composition is a vaccine adjuvant for enhancing or modulating an immune response against the mycobacterium when used with a vaccine against the mycobacterium in a mammal.

In a preferred embodiment, the isolated mycobacterium membrane vesicles are M. tuberculosis membrane vesicles. In an embodiment, the mycobacterium is an H37Rv strain M. tuberculosis.

In an embodiment, the isolated mycobacterium membrane vesicles are M. bovis Bacille Calmette Guerin (BCG) Pasteur membrane vesicles. In an embodiment, the isolated mycobacterium membrane vesicles are from Mycobacterium avium, Mycobacterium bovis, Mycobacterium diphtheria, Mycobacterium intracellulare, Mycobacterium leprae, Mycobacterium lepraemurium, Mycobacterium phlei, Mycobacterium smegmatis, Mycobacterium fortuitum, Mycobacterium lufu, Mycobacterium paratuberculosis, Mycobacterium habana, Mycobacterium scrofulacium, or Mycobacterium kansasii.

In an embodiment, the composition further comprises a tuberculosis vaccine active agent. The active agent may be a mycobacterium, for example an attenuated mycobacterium, or a mycobacterium subunit, for example of a M. tuberculosis. In an embodiment, the active agent is an H1 or H4 subunit vaccine or comprises Ag85B antigen. In an embodiment, the tuberculosis vaccine active agent is a M. bovis BCG Pasteur, or a portion thereof.

The invention also provides a method of eliciting an immune response in a subject comprising administering to the subject any of the vesicle compositions described herein an amount effective to elicit an immune response. In an embodiment, the immune response comprises a Th1 response.

The invention also provides a method of improving the efficacy of an anti-mycobacterial immunization administered to a subject, comprising administering to the subject any of the compositions described herein in an amount effective to improve efficacy of an anti-mycobacterial immunization administered to a subject.

In an embodiment, the anti-mycobacterial immunization is an anti-tuberculosis immunization. In an embodiment, the tuberculosis vaccine active agent is a M. tuberculosis BCG Pasteur, or a portion thereof. In an embodiment, the composition is administered to the subject via the same route as the anti-mycobacterial immunization is administered. In an embodiment, the composition is administered subcutaneously.

The invention also provides a method of immunizing a subject against tuberculosis comprising administering to the subject an amount of any of the instant compositions in an amount effective to immunize a subject against tuberculosis.

In an embodiment, the subject is immunocompromised. In an embodiment, the composition is administered subcutaneously.

Also provided is a composition comprising a plurality of isolated bacterium membrane vesicles isolated from a pathogenic bacterium that produces membrane vesicles. In an embodiment, the composition further comprises an immunological adjuvant. In an embodiment, the composition further comprises a pharmaceutically acceptable carrier.

Also provided is a method of eliciting an immune response in a subject comprising administering to the subject any of the instant compositions in an amount effective to elicit an immune response.

Also provided is a method of preparing an isolated mycobacterium membrane vesicles preparation comprising centrifuging a culture of mycobacterium and treating the resultant product so as to remove live mycobacteria and recover the vesicles, thereby preparing the isolated mycobacterium membrane vesicles preparation. In an embodiment, the isolated mycobacterium membrane vesicles are M. tuberculosis membrane vesicles. In an embodiment, the isolated mycobacterium membrane vesicles are purified. In an embodiment, the isolated mycobacterium membrane vesicles are admixed with a pharmaceutically acceptable carrier.

In an embodiment, the isolated mycobacterium membrane vesicles are M. bovis Bacille Calmette Guerin (BCG) Pasteur membrane vesicles. In an embodiment, the isolated mycobacterium membrane vesicles are from Mycobacterium avium, Mycobacterium bovis, Mycobacterium diphtheria, Mycobacterium intracellulare, Mycobacterium leprae, Mycobacterium lepraemurium, Mycobacterium phlei, Mycobacterium smegmatis, Mycobacterium fortuitum, Mycobacterium lufu, Mycobacterium paratuberculosis, Mycobacterium habana, Mycobacterium scrofulacium, or Mycobacterium kansasii. In an embodiment, the mycobacterium is an H37Rv strain M. tuberculosis.

In an embodiment of the inventions described herein, the mycobacterium vesicles are mycobacterium tuberculosis vesicles, and the mycobacterium tuberculosis is one of the following: Mycobacterium tuberculosis H37Rv, BTB05-552, BTB05-559, CDC1551, CTRI-2, F11, H37, H37Ra, HN878, KZN 1435, KZN 4207, KZN R506, KZN V2475, R1207, RGTB327, S96-129, X122, ‘98-R604 INH-RIF-EM’, 02_{—}1987, 210, 94_M4241A, C, CDC1551A, CPHL_A, CTRI-4, EAS054, GM 1503, K85, KZN 605, OSDD071, OSDD504, OSDD518, SUMu001, SUMu002, SUMu003, SUMu004, SUMu005, SUMu006, SUMu007, SUMu008, SUMu009, SUMu010, SUMu011, SUMu012, T17, T46, T85, T92, W-148, str. Haarlem, 210_{—}16C2_{—}24C1, 210_{—}16C2_{—}24C2, 210_{—}32C4, 210_{—}4C15, 210_{—}4C15_{—}16C1, 210_{—}4C15_{—}16C1_{—}48C1, 210_{—}4C15_{—}16C1_{—}48C2, 210_{—}4C15_{—}16C1_{—}56C1, 210_{—}4C15_{—}16C1_{—}56C2, 210_{—}4C31, 210_{—}4C31_{—}16C1, 210_{—}4C31_{—}16C1_{—}24C1, 210_{—}4C31_{—}16C1_{—}40C1, 210_{—}4C31_{—}16C2, 210_{—}8C1, 210_{—}8C6, BC, CTRI-3, H37Rv_{—}2009, NJT210GTG, str. Erdman=ATCC 35801, str. Erdman WHO, CCDC5079, CCDC5180, RGTB423, UT205, CTRI-1, H37RvAE, H37RvCO, H37RvHA, H37RvJO, H37RvLP, H37RvMA, LAM7, NCGM2209, RGTB306, WX1, WX3, XDR1219, XDR1221, str. Beijing/W BT1, or str. Erdman (ATCC 35801). In a preferred embodiment, the mutant is a mutant of M. tuberculosis H37Rv strain.

In an embodiment, wherein the composition is intended for administration to a subject, the composition can further comprise an immunological adjuvant. Immunological adjuvants encompassed within the compositions and methods of the invention are widely known in the art and include alum, other aluminum salts (e.g. aluminum phosphate and aluminum hydroxide) and squalene. Other immunological adjuvants encompassed within the compositions and methods of the invention include the compounds QS21 and MF59. In an embodiment, the composition is vaccine. In an embodiment, the vaccine comprises a live attenuated mycobacterium. In an embodiment, the vaccine comprises a portion of a mycobacterium but does not comprise the whole of the mycobacterium. In an embodiment, the vaccine comprises a pharmaceutically acceptable carrier. In an embodiment, the vaccine further comprises an immunological adjuvant.

Any of the compositions of the invention can be used to evoke an immune response in a subject. In an embodiment, administration of a composition of the invention, or the isolated mycobacteria membrane vesicles of the invention, is used to elicit an immune response in the subject. In an embodiment, the eliciting an immune response in a subject is effected by a method comprising administering to the subject the composition or isolated mycobacterium vesicles in an amount effective to elicit an immune response.

In a preferred embodiment of the mutants, compositions and methods of the invention, the mutant is a mutant M. tuberculosis H37Rv strain. For H37Rv genome, see NCBI Reference Sequence: NC_{—}000962.2, see GenBank: AL123456.2.

The methods disclosed herein involving subjects can be used with any species capable of being infected by mycobacteria, preferably M. Tuberculosis. In a preferred embodiment, the subject is a mammalian subject. Most preferably, the mammal is a human.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

Here, it was investigated whether systemic administration of MV preparations could induce protective immune against an Mtb pulmonary infection. It is demonstrated that systemic administration of isolated mycobacterial vesicles elicits a protective response protection against an Mtb aerosol infection.

Mycobacterial culture: Mycobacteria (Mycobacterium bovis Bacillus Calmette-Guerin (BCG, Pasteur strain), M. tuberculosis strains H37Rv (Mtb) and M. smegmatis) were grown in a minimal medium (MM) consisting of KH₂PO₄ 1 g/l, Na₂HPO₄ 2.5 g/l, asparagine 0.5 g/l, ferric ammonium citrate 50 mg/l, MgSO₄×7 H₂O 0.5 g/l, CaCl₂ 0.5 mg/l, ZnSO₄ 0.1 mg/l, with or without Tyloxapol 0.05% (v/v), containing 0.1% (v/v) glycerol, pH 7.0. The M. bovis BCG strain lacking the 19 kDa protein (D19) was a gift from D. Young. This strain was grown in the presence of 50 μg/ml of hygromicin. Cultures were grown for up to 10 days for slow growers and 4 days for fast growers in roller bottles at 37° C. In some experiments as stated, cultures were grown in Middlebrook 7H9 medium (M7H9) supplemented with 10% (v/v) OADC enrichment (Becton Dickinson Microbiology Systems, Spark, Md.), 0.5% (v/v) glycerol and with or without Tyloxapol 0.05% (v/v; Sigma). M. tuberculosis H37Rv subcellular fractions were prepared as described (15).

Vesicle isolation and purification: Vesicles were isolated as described (7). Briefly, cells were pelleted (3,450 g for 15 min, 4° C.) from 1000 ml cultures and the supernatants were filtered through a 0.45-mm-pore size polyvinylidene difluoride filter (Millipore, Billerica, Mass.). The supernatant volumes were then concentrated approximately 20-fold using an Amicon (Millipore) ultrafiltration system with a 100 kDa exclusion filter. The concentrate was then sequentially centrifuged at 4,000 and 15,000 g (15 min, 4° C.) to remove cell debris and aggregates and the remaining supernatant was then centrifuged at 100,000 g for 1 h at 4° C. to sediment the vesicular fraction into a pellet. The supernatant was then discarded, the pellet was suspended in 1 ml of 10 mM HEPES, 0.15 M NaCl and mixed with 2 ml of Optiprep solution (Sigma) in 10 mM HEPES and 0.15 M NaCl (yielding 35% (w/v) Optiprep final concentration). The crude vesicle sample was then overlaid with a series of Optiprep gradient layers with concentrations ranging from 30-5% (w/v). The gradients were centrifuged (100,000×g, 16 h) and 1 ml fractions were removed from the top. The fractions were then dialyzed separately in PBS overnight and again recovered by sedimentation at 100,000×g for 1 h. Finally, the vesicles were suspended in LPS-free PBS. Vesicles quantitation was performed using the BCA protein assay (Thermo Scientific, Rockford, Ill.).

Immunizations and Mtb challenge: Six to 8-week-old female wild type (C57BL/6) mice were purchased from the National Cancer Institute (NCI, Frederick, Md.). All mice were maintained in specific pathogen-free conditions, and were transferred to biosafety level 3 conditions for infection with M. tuberculosis. All procedures involving the use of animals were in compliance with protocols approved by the Albert Einstein College of Medicine Institutional Animal Use and Biosafety Committees. BCG cultures were grown to mid-log phase, washed, suspended in PBS+0.05% Tyloxapol (PBS/T) and sonicated before infection. Mice were vaccinated with BCG subcutaneously (SC) at the scruff of the neck with 1×10⁶ CFU in 100 μl for four or eight weeks. Approximately 2.5 micrograms of vesicles were administered per mouse intraperitoneally (IP) or subcutaneously (SC) in a final volume of 200 μl PBS. In the boosting experiments the vesicles were given on top of BCG after 4 weeks IP or SC. For aerosol challenge with Mtb, a low-density freezer stock of H37Rv was grown in 7H9 liquid medium to an A600 of 0.4-0.8. 2×10⁶ CFU ml−1 of bacteria in PBS/T plus 0.04% (vol/vol) Antifoam Y-30 (Sigma) was placed in a nebulizer attached to an airborne infection system (University of Wisconsin Mechanical Engineering Workshop). Mice were exposed to aerosol for 40 min, during which approximately 100 bacteria were deposited in the lungs of each animal. Tissue bacterial loads in tissues for aerosol infections were determined by plating organ homogenates onto 7H11 agar OADC plates. Colonies were counted after 21 d of incubation at 37° C.

ELISA: Vaccinated mice were bled by an orbital bleed. Serum was collected by centrifugation at 10,000×g for 20 min and stored at −20° C. until use. Serum reactivity was measured by ELISA against a whole cell lysate preparation of Mtb. ELISA plates (96 wells) were coated with 20 μg ml−1 Mtb lysate in PBS and incubated at 37° C. for 1 h and then blocked in PBS+3% BSA for 1 h at 37° C. or overnight at 4° C. Wells were washed three times in PBS with 0.05% (PBS/T) Tween-20 and then incubated with a 1:300 diluted mouse serum for 1 h at 37° C. After washing three times with PBS/T wells were incubated with an alkaline-phosphatase conjugated goat antibody to mouse immunoglobulin G (Southern biotechnologies) for 1 h at 37° C. Color was developed with p-nitrophenyl phosphate. The titer was defined as the dilution of sera resulting in an A405 2 times greater than the background.

Immunoblot: Serum reactivity against an Mtb lysate was also tested by immunobloting (16). To find the identity of the 19 kDa band, serum reactivity was also measured against a whole cell lysate preparation of a Δ19 kDa mutant strain of BCG. Electrophoresis was done with a 12% resolving gel at 100 V. Gels were transferred to nitrocellulose membranes, which were then blocked overnight in a buffer containing 5% milk in PBS with 0.1% Tween 20. Individual channels on a blotting frame were incubated with diluted serum (1:400) sera for 1 h at room temperature or overnight at 4° C. Channels were washed three times with blocking buffer and then incubated with a horseradish peroxidase-conjugated goat antibody to mouse immunoglobulin G (Sigma) for 1 h at RT. Membranes were developed using luminol (Pierce).

IFNγ ELISPOT assay: Splenocytes were cultured in ELISPOT plates (1×10⁶/well; Millipore, Danvers, Mass.) coated with IFNγ capture antibody (clone R4.6A2; BD Biosciences). Mtb sonicate (BEI Resources, VA) was added (20 μg/ml) and plates were for 24 h at 37° C. After removal of cells, plates were washed with PBS followed by PBS with 0.05% Tween 20 (PBST). Biotinylated anti-IFNγ detection antibody (clone 4S.B3; BD Biosciences) was added for 2 h at 37° C., followed by washing with PBST. Streptavidin-alkaline phosphatase (Sigma-Aldrich) was added to the plates for 1 h (37° C.), followed by washing and addition of BCIP/NBT substrate (Sigma-Aldrich). The reaction was stopped by washing the wells with water, and spots were counted using an ELISPOT reader (Autoimmun Diagnostika, Strasberg, Germany).

Intracellular cytokine analysis: For analysis of cytokine-producing CD4+ T and CD8+ T cells, spleen cell suspensions were isolated and placed in 96-well plates in RPMI-1640 with 10% FCS. The samples were stimulated with 10 μg ml−1 a whole cell lysate Mtb preparation (BEI Resources, VA) plate-bound monoclonal antibody to mouse CD3ε (clone 145-2C11), with unstimulated wells serving as negative controls. Samples were combined with 1 μg ml−1 soluble antibody to mouse CD28 (clone 37.51). After 2 h at 37° C., 10 μg ml−1 of Brefeldin-A (Sigma) was added to all samples, followed by incubation for 4 h. Cells were stained with Blue LIVE/DEAD viability dye (Invitrogen) followed by antibody to FcγRI/III (clone 2.4G2; American Type Culture Collection), with fluorochrome-conjugated monoclonal antibodies for surface staining: antibody to CD3ε (clone 145-2C11; eBioscience), antibody to CD44 (clone IM7; eBioscience), antibody to CD8α (clone 53-6.7; BD Bioscience) and antibody to CD4 (clone GK1.5; BD Bioscience). Cells were fixed with 2% (vol/vol) paraformaldehyde, washed with permeabilization buffer (PBS with 1 mM Ca²⁺, 1 mM Mg²⁺, 1 mM HEPES, 2% (vol/vol) FCS and 0.1% (wt/vol) saponin) and then blocked in permeabilization buffer plus 5% (vol/vol) normal mouse serum (Jackson ImmunoResearch Laboratories). Intracellular cytokines were detected with fluorochrome-conjugated antibodies to IL-2 (clone JES6-5H4; eBioscience), IFN-γ (clone XMG1.2) and TNF-α (MP6-XT22) (both from BD Biosciences). Data were acquired on an LSR II flow cytometer (BD Biosciences), and data analysis was performed using FlowJo software (Tree Star).

Histology: Lungs and spleens were removed and fixed in 10% neutral buffered formalin (Fisher Scientific, Fair Lawn, N.J.). Tissues were embedded with paraffin, sectioned at 5 μm thickness, and stained with hematoxylin and eosin and by Kinyoun's acid fast (AF) stain. Area of lung lesions was calculated in Image J and expressed as percentage of total lung area. Five different lung sections per mouse were counted.

### Experimental Results

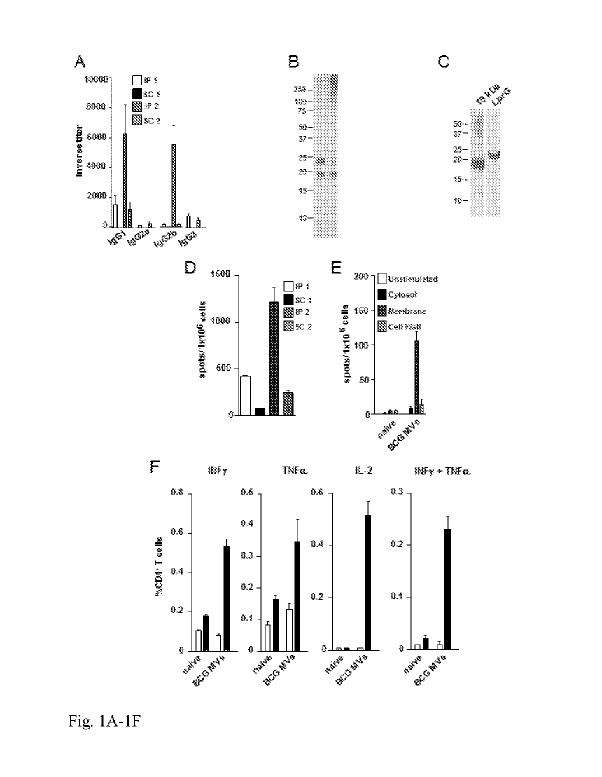

In an attempt to characterize the immune response, if any, of MVs in mice we immunized C57BL/6 mice with 2.5 μg of BCG, H37Rv and M. smegmatis MVs (based on protein concentration) intraperitoneally (IP) with no adjuvant. After 30 d the serum antibody response was measured by ELISA against a whole cell Mtb lysate (FIG. 1A). In each experiment PBS, IP-injected mice were used as a control. No detectable levels of IgGs and low levels of IgM (1:300) were found in the BCG immunized mice. The antibody response was different among the MVs tested Immunization with BCG MVs produced the more diverse antibody response with all the IgG isotypes detected and significant levels of IgG1, IgG2b and IgG3. H37Rv MVs immunization triggered an antibody response dominated by IgG1 and IgG2b and IgM was detected only after M. smegmatis MVs immunization (FIG. 1A). The type of Mtb proteins recognized by antibodies from two mice immunized via IP with different MVs by immunoblot was investigated (FIG. 1 B). BCG immunization generated strong IgG responses to a protein with an estimated molecular mass of 24 KDa (FIG. 1B), which may correspond to LprG (Rv 1411c) (17, 18) (FIG. 1C). When sera from BCG or H37Rv MVs immunized mice were tested an additional band with an estimated mass of 19 kDa was detected (FIG. 1B). This protein may correspond to LpqH, which has been detected in purified mycobacterial MVs (7) and has an estimated molecular mass of 19 kDa (FIG. 1C) (18). Interestingly, the differences in the reactivity to the 19 kDa and 24 kDa proteins by sera from BCG and H37Rv MVs immunized (FIG. 1B) mice suggests that the amount of these proteins vary in the different types of vesicles.

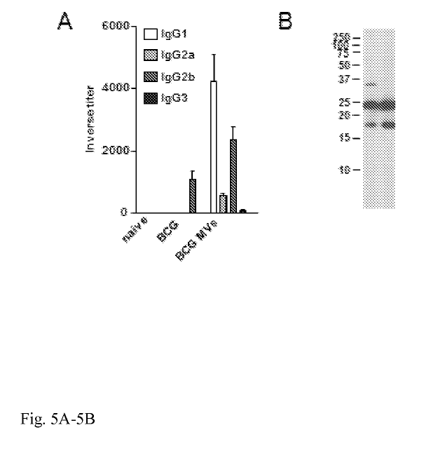

Since lipoproteins are located at the mycobacterial cell membrane (18), and MVs originate primarily from this cellular compartment (7), antibodies from BCG or H37Rv MVs immunized sera should preferentially recognize the membrane fraction of M. tuberculosis. When ELISA was performed on plates coated with a mycobacterium membrane fractions the measured serum titers were significantly greater (FIG. 5). Detectable levels of IgG2b were observed in serum from BCG immunized mice. Levels of IgM also increased in all the immunized samples. Levels of IgG1 and IgG2b were four-fold higher in BCG and H37Rv MVs treated mice (FIG. 5). These results show that much of the antibody response is directed to the mycobacterial membrane. Moreover, immunoblot analysis of the same serum samples against an Mtb membrane fraction also showed an increased reactivity (FIG. 5) compared to that of whole cell lysate (FIG. 1B) and additional bands of ˜37 kDa were detected. The differences in reactivity to 19 and 24 kDa proteins were strongly evident when sera from BCG and H37Rv MVs immunized mice were compared. No additional bands were detected in sera from BCG vaccinated mice. These data strongly suggest immunization with mycobacterial MVs triggered a humoral response that reacted with the mycobacterial cellular membrane, and specifically with lipoproteins. No strong response was found upon M. smegmatis vaccination indicating that it does not promote the production of specific antibodies against Mtb.

Mycobacteria MVs induce a high Th1 response: A T helper type 1 (Th1) cell response is crucial in protective immunity to many intracellular pathogens and Th1-polarized responses play an important role in the control of Mtb infection (19). As above, mice were immunized with a single dose of 2.5 μg of BCG, H37Rv or M. smegmatis MVs via IP and total T cell responses on ex vivo Mtb sonicate-stimulated spleens were studied by IFNγ ELISPOT at day 30. Among the MVs tested, the H37Rv MVs triggered the highest T cell response, being two-fold higher than that elicited by BCG MVs and almost on 1.5-fold that observed with BCG immunization (FIG. 1D). Similar levels to the naïve and unstimulated treatments of IFNγ producing splenocytes were detected after M. smegmatis MVs immunization suggesting that these MVs do not produce Mtb specific T cell responses (FIG. 1D).

Next, it was investigated whether the splenic responses obtained upon MVs stimulation were also biased for the mycobacterial membrane fraction, as occurred with antibody responses. Since there was not access to specific vesicle associated antigens like the lipoproteins 19 kDa or LprG, it was not practical to dissect T cell responses as done with antibodies (FIG. 1C). Nevertheless, since vesicles are enriched in lipoproteins, and lipoproteins are localized at the cell membrane (18), the same experiments were performed by stimulating with a membrane fraction of Mtb as well as other subcellular fractions such as cell wall or cytosol. The stimulation with membrane fraction induced the highest T cell response in BCG splenocytes (FIG. 1E), suggesting that membranes proteins may determine T cell antigenicity. Similar levels of IFNγ producing cells were detected upon cell wall fraction stimulation. Almost two-fold less potent responses were detected in BCG MVs splenocytes but, again, the highest stimulation capacity was exclusively produced by stimulation with an Mtb membrane fraction (FIG. 1E). A more heterogeneous response was obtained in H37Rv MVs splenocytes where the membrane and cell wall fractions triggered two-fold higher responses relative to those elicited by BCG. These results indicate that despite the protein heterogeneity in vesicle composition, only a minor fraction of these proteins determines the immunogenicity of vesicles in terms of antibodies and T cells. Moreover, the response triggered by the current BCG vaccine seems to be directed to membrane antigens. Again, immunization with M. smegmatis MVs did not induce a significant and specific T cell response on spleen (FIG. 1E).

Next, the multifunctionality of the CD4+ T cell in response in MVs-immunized mice was examined Mice were immunized as above and cytokine production by splenic CD4+ T cells was analyzed at day 30. Upon re-stimulation in vitro with an Mtb sonicate, a substantial increase in INFγ, TNFα and IL-2 production was observed between naive and BCG or MVs-immunized mice with no remarkable differences (FIG. 1F). Only a increase in IFNγ producing cells was detected upon H37Rv MVs immunization (FIG. 1F). A single dose of MVs and vaccination with BCG only triggered the multifunctional cell population of CD4+T cells producing IFNγ and TNFα. No detectable levels of cytokines producing cells were found when mice were vaccinated with M. smegmatis MVs in agreement with the lack of T cells responses previously described above (FIG. 1E). These results indicate that stimulation of mice with BCG or H37Rv MVs produced a strong Th1 response similar to the one produced by the current vaccine BCG.

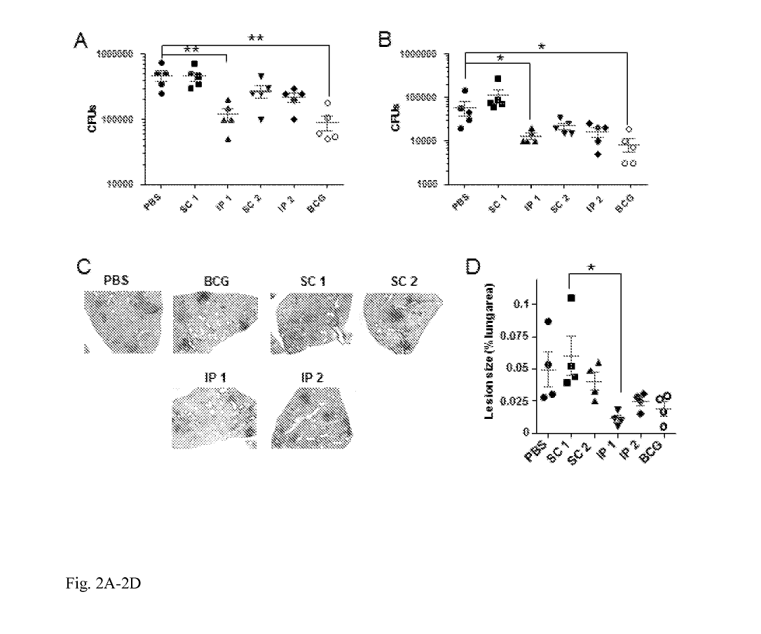

Mycobacterial vesicles induce protective immune responses in mice: To test the protective efficacy of MV, mice were immunized with 2.5 μg of BCG MVs via IP. Mice were challenged 4 weeks later with a low dose (50-100 bacilli) of Mtb via aerosol and CFUs were determined at 4 weeks after infection in lungs and spleens. For controls mice were vaccinated with either one million of bacilli of BCG Pasteur strain or sham (PBS) immunized. It was found that immunization of mice with BCG MVs without adjuvant was associated with marked reductions in lung CFUs comparable to those of mice immunized with BCG (FIG. 2A). These results were also true for bacterial burden levels in spleen (FIG. 2B). Histopathological analysis revealed evidence of robust immunity in MVs-vaccinated mice. At day 28 of infection, PBS treated mice manifested severe diffuse granulomatous pneumonia (FIG. 2C). Although lesion sizes were similar in mice vaccinated with BCG and MVs, a significant decrease in the lesion size observed in lungs from MVs treated mice (FIG. 2D). One of the desired properties of any pre-exposure anti-tuberculosis vaccine is the prevention of TB reactivation (2). Therefore, vaccines need to promote a long-lasting protection. In this context bacterial burden was determined in lungs and spleen of vaccinated mice after nine weeks of infection. Interestingly, immunization with BCG MVs was no longer protective at the later time (FIG. 2E). A group of mice vaccinated with H37Rv MVs was also analyzed. Remarkably, these mice showed significantly lower lung CFUs compared with BCG vaccinated mice. In agreement with the previous results vaccine responses, M. smegmatis MVs did not promote protection (FIG. 2E). A similar trend was observed when spleens were analyzed for bacterial counts (FIG. 2F), indicating that vaccination with BCG or H37Rv MVs reduced extrapulmonary dissemination of the bacteria.

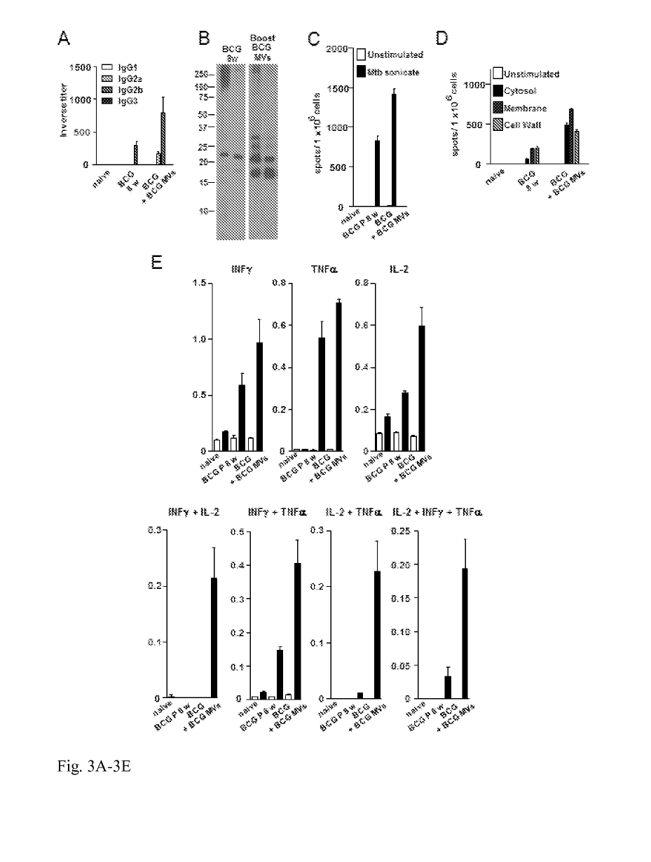

Boosting BCG vaccine with mycobacterial vesicles: the ability of mycobacterial MVs to boost BCG vaccine was investigated. Consequently, BCG vaccinated mice were boosted at 2 months with a single dose of BCG, H37Rv and M. smegmatis MVs and after two weeks Mtb-specific antibodies and T cell responses were analyzed in serum and spleen, respectively. The response was compared to that of mice vaccinated with BCG alone for three months and sham (PBS) vaccinated mice. A low antibody response including IgM and IgG1 was detected in serum of BCG-vaccinated mice (FIG. 3A). When animalas were boosted with BCG MVs the antibody titers increased almost 8-fold for IgM and new levels of IgG2a and IgG2b were detected. A similar response to BCG MVs sera was obtained upon re-stimulation with H37Rv MVs, but with additional levels of IgG1. The response obtained when BCG vaccinated mice were boosted with M. smegmatis MVs was similar to H37Rv MVs mice but with additional levels of IgG3 (FIG. 3A). Serum reactivity to Mtb antigens of vaccinated mice was also analyzed by immunoblotting. As in sera analyzed at 4 weeks, one single band with an estimated molecular weight of 24 kDa was detected in serum from two different BCG-vaccinated mice (FIG. 3B). This profile changed in serum from BCG or H37Rv MVs boosted mice. In addition to the 24 kDa antigen other proteins with an estimated mass of 19 kDa, 30 kDa and 35 kDa were detected (FIG. 3B), indicating that MVs immunization promoted diversity in the antigens recognized.

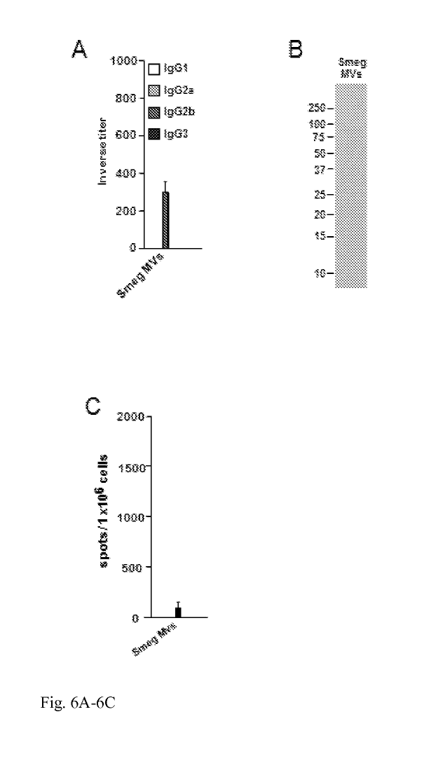

As in single vesicle stimulation experiments (FIG. 1B, C), antigens with 19 kDa and 24 kDa may correspond to lpqH and LprG proteins. The reactivity of M. smegmatis MVs sera was poor compared to other MVs. This result shows that boosting BCG vaccination with BCG or H37Rv MVs elicited a stronger and more diverse response, including antigens previously detected in single immunization experiments and new ones. Moreover, both isotype diversity and serum titers increased in MVs samples when reactivity was tested to an Mtb membrane fraction. When mice were boosted with M. smegmatis MVs there was no increase in antibody titers over that resulting from BCG vaccination (FIG. 6).

BCG or H37Rv MVs boosting increased 1.5-fold the number of Mtb specific splenic T cells compared to BCG vaccinated mice (FIG. 3C). Similar levels of IFNγ producing T cells were found upon M. smegmatis MVs stimulation as with BCG immunization, indicating the poor capacity of these vesicles to boost the prior immune response. Stimulation of same splenocytes with Mtb subcellular fractions including cell wall, cytosol and membrane showed a balanced T cell response in BCG vaccinated mice (FIG. 3D). When BCG-vaccinated mice were boosted with BCG or H37Rv MVs higher responses were obtained and more specific T cells were detected upon Mtb membrane stimulation (FIG. 3D). The T cell responses elicited by BCG vaccination did not change when mice were boosted with M. smegmatis MVs. The quality of the T cell response in the spleen at 4 weeks was determined by intracellular cytokine staining of splenocytes as above (FIG. 1F). Analysis of single cytokines in CD4+ T cells showed an increase in TNFα, IL-2 and IFNγ in animals boosted with BCG or H37Rv MVs compared to BCG. This increase was statistically significant for IFN and IL-2 in groups receiving H37Rv MVs. In addition, the level of CD4+ T cells producing multiple cytokines also increased upon vesicle boosting. Remarkably, no INFγ+IL-2 positive cells and low levels of IL-2+TNFα and IFNγ+TNF positive cells were detected in the spleens of BCG vaccinated mice. The amount of CD4+ T cells with ability to produce three of them was again higher in spleens from MVs boosted mice (FIG. 3F). This data indicate that priming BCG response with vesicles enhance the quality of the immune response. In contrast, vaccination with M. smegmatis MVs did not change the BCG cytokines levels (FIG. 3E).

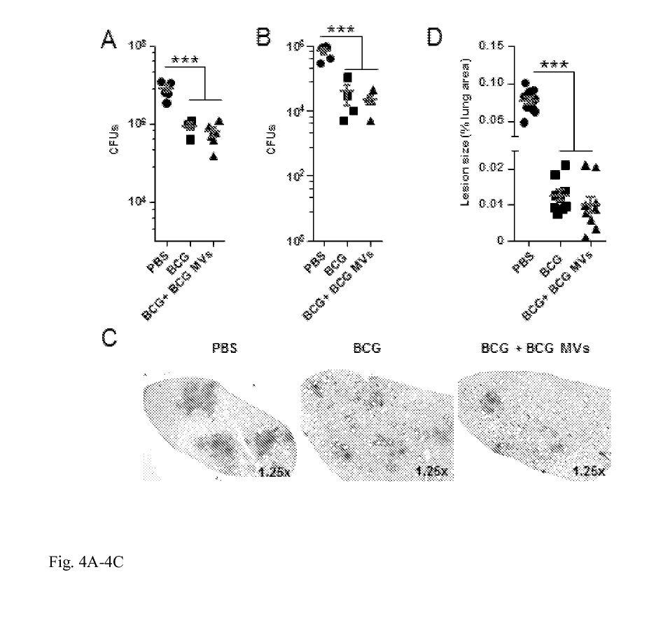

Four weeks after boosting with BCG MVs mice were challenged with a low dose of Mtb via aerosol and lung and spleen mycobacterial counts were enumerated 4 weeks after challenge. All mice in the BCG-vaccinated groups had significantly fewer bacilli in the lungs than the control mice (1.12±0.04 log 10 CFU reduction, P<0.001). BCG MVs-boosted mice had slightly lower bacterial counts than the BCG-vaccinated mice (1.24±0.08 log 10 CFU reduction, P<0.001) (FIG. 4A). This was also true for the bacterial counts in spleen with a mean reduction in bacterial burden of 1.2±0.09 (P<0.001) and 1.55±0.02 (P<0.001) in BCG and MVs-vaccinated mice, respectively (FIG. 4B).

In agreement with these results, the lung histological analysis showed a marked reduction in inflammation between PBS and BCG or BCG MVs-vaccinated mice (FIG. 4C). Indeed, both BCG and MVs-boosted mice showed a reduction in the size of lesions, suggesting an enhanced control of the bacterial burden. Lesions from BCG MVs boosted mice were smaller than those of BCG-vaccinated mice, but the differences were not statistically significant (FIG. 4D). Analysis of CFUs at 9 weeks did not show differences in lung CFUs between BCG vaccinated mice and boosted mice including all type of MVs (FIG. 4E). However, when CFUs levels were compared to PBS, the group of animals boosted with H37Rv MVs demonstrated better protection efficacy. Interestingly, this group was the only one, along with the BCG vaccinated mice, showing lower CFUs in the spleen (FIG. 4D). These results show that boosting BCG vaccinated mice with BCG MVs produces a short term protection efficacy that does not worsen the previous response. The robustness of this response was maintained in mice immunized with H37Rv MVs.

### DISCUSSION

The elucidation of the M. tuberculosis genome highlighted a remarkable capacity for lipid biosynthesis (20). Subsequent studies have shown that many genes involved in lipid biosynthesis are essential for virulence in animal models. Recently, two independent groups have described the Mtb lipidome using high resolution mass spectrometry (21, 22). Despite these advances, little is known about the mechanisms by which Mtb release lipids to the extracellular milieu. This laboratory has recently shown that M. tuberculosis, and other mycobacterial strains, including the vaccine strain BCG and the environmental strain M. smegmatis, produce membrane vesicles that are released to the extracellular space (7). Remarkably, only vesicles from the virulent strains are enriched in lipoproteins, which are well known TLR2 agonists involved in modulation of host immune response (17). Lipid analysis indicated prevalence for polar lipids, consistent with origin from the cellular membrane. Intratracheal administration of vesicles to mice followed by aerosol challenge resulted in an increase of bacterial burden in the lungs (7), suggesting a Koch phenomenon where an immune response to the vesicles in the lung worsened the outcome of infection. The contribution of bacterial vesicles to virulence has been demonstrated for other bacterial pathogens, including B. anthracis (12), N. meningitidis (23), E. coli (3) or P. aeruginosa (3). However, when these vesicles were tested as vaccine preparations and administered systemically they elicited immune responses that were associated with significant protection. Moreover, the immune response of bacterial MVs-based vaccines neutralizes bacterial toxins or is directed to the bacteria surface and membrane antigens, promoting bacterial opsonization and complement-mediate killing.

In this report, isolated MVs from three mycobacterial strains: BCG, H37Rv M. tuberculosis and M. smegmatis were studies as an anti-tuberculosis vaccine. Both antibodies and T cells appeared to be responsible for the protective efficacy of BCG and H37Rv MVs. Furthermore, antibodies were preferentially produced to membrane compounds, and specifically to lipoproteins LprG and LpqH, suggesting that they may determine the mycobacterial MVs antigenicity. Enhanced antibody reactivity to a mycobacterial membrane fraction confirmed this hypothesis. Moreover, when antigenicity of serum BCG vaccinated mice was tested, a unique band, possibly corresponding to LprG, was detected, indicating that lipoproteins strongly determine antigenicity in the current anti-tuberculosis vaccine. Interestingly, a global analysis of antibody response to Mtb reported that LprG is among the most reactive proteins (24). M. tuberculosis uses lipoproteins such as LpqH or LprG to inhibit antigen processing and presentation, induce proinflammatory or inhibitory cytokines, and control of costimulatory molecule expression in APCs leading to the modulation of T cell function (17, 25, 26). Thus, it is believed that specific antibodies produced upon mycobacterial MVs vaccination may interact with lipoproteins inactivating or neutralizing their immunomodulatory effect. Interestingly, LprG and LpqH were recently shown to activate and module CD4+T cells effector functions in a TLR2 dependent manner, independently from antigen presenting cells, suggesting that M. tuberculosis lipoproteins can regulate adaptive immunity not only by inducing cytokine secretion and costimulatory molecules in innate immune cells but also through directly regulating the activation T lymphocytes (27). Moreover, this finding indicates that mycobacterial MVs can serve as an adjuvant since these molecules directly enhance CD4+ T cell memory responses. Recently, some mycobacterial lipids associated to MVs, such as PIM2 or PIM6, were used as an adjuvant showing their capacity to modulate the immune response and efficacy of a protective vaccine against M. bovis infection (28)

The enhanced splenic T cell responses, including higher total T cell responses and increased levels of multifunctional T cells of MVs-vaccinated mice, indicate that they may also contribute to the protection efficacy. In agreement with antibody studies responses were also enhanced after stimulation with M. tuberculosis membrane and cell wall fractions. The protective host response to mycobacterial infection is believed to be mainly mediated by a cell-based immunity (29). On the other hand, antibody responses are generally believed to have no protective role (30) despite considerable evidence that protective antibodies can be generated against M. tuberculosis and that B cells contribute to host protection (31-33). Remarkably, most of the current subunit vaccines in clinical trials are composed of immunodominant antigens like ESAT-6, Ag85b, and Ag85A, each of which have been shown to produce mixed humoral and cellular responses (34-37).

The necessity of a long-term protection in anti-tuberculosis vaccines is likely to be an important quality to prevent reactivation of latent infection. This does not seem to be the case for BCG MVs using the present scheme of immunization and mice may require several doses to keep the immunization status to control the infection, as it is true for most of the subunit vaccines (34-37). On the other hand, immunization with a single dose of H37Rv MVs promoted containment of bacilli in lungs at later time points. Although BCG and H37Rv MVs have a similar protein composition (7), subtle differences may determine their differential contribution to protection. Consistently, a differential recognition for LprG and LpqH was found between these two sets of MVs. It is also noted that this immune response was obtained with MVs preparations alone without the presence of adjuvant Ongoing experiments including multiple doses and co-administration with adjuvants will elucidate the potential capacity of MVs to enhance the current vaccine BCG.

The fact that almost 80% of the current pre-exposure vaccines in clinical trials are subunit booster vaccines to be given on top of an initial BCG prime suggests the necessity of improving the immune response triggered by BCG (2). Analysis of the immune response of BCG vaccinated and MVs-boosted mice showed an increase in antibody titers and the levels of all CD4+ T cells producing single and multiple cytokines increase in BCG and H37Rv boosted mice. Although BCG MVs administration did not reach a higher level of protection when given on top of a BCG vaccine at 4 weeks, their administration did not worsen the previously established immune response. However, re-stimulation with H37Rv MVs promoted a more robust protective efficacy at later time points after challenge. These observations indicate that MVs preparations have considerable promise as vaccine components alone and/or in combination with BCG. This study shows mycobacterial MVs as an alternative vaccine to BCG in protection against Mtb in mice. This is first effective anti-tuberculosis vaccine containing lipoproteins.

### REFERENCES

1. WHO (2011) World Health Organization. Global Tuberculosis Control: WHO Report. WHO Library Cataloguing-in-Publication Data 2011.
2. Kaufmann S H (2010) Future vaccination strategies against tuberculosis: thinking outside the box. Immunity 33(4):567-577.
3. Kuehn M J & Kesty N C (2005) Bacterial outer membrane vesicles and the host-pathogen interaction. Genes Dev 19(22):2645-2655.
4. Oliveira D L, et al. (2010) Biogenesis of extracellular vesicles in yeast: Many questions with few answers. Commun Integr Biol 3(6):533-535.
5. Oliveira D L, et al. (2010) Characterization of yeast extracellular vesicles: evidence for the participation of different pathways of cellular traffic in vesicle biogenesis. PLoS One 5(6):e11113.
6. Rodrigues M L, et al. (2008) Extracellular vesicles produced by Cryptococcus neoformans contain protein components associated with virulence. Eukaryot Cell 7(1):58-67.
7. Prados-Rosales R, et al. (2011) Mycobacteria release active membrane vesicles that modulate immune responses in a TLR2-dependent manner in mice. J Clin Invest 121(4):1471-1483.
8. Hoist J, et al. (2009) Properties and clinical performance of vaccines containing outer membrane vesicles from Neisseria meningitidis. Vaccine 27 Suppl 2:B3-12.
9. Fransen F, et al. (2010) Differential effect of TLR2 and TLR4 on the immune response after immunization with a vaccine against Neisseria meningitidis or Bordetella pertussis. PLoS One 5(12):e15692.
10. Alaniz R C, Deatherage B L, Lara J C, & Cookson B T (2007) Membrane vesicles are immunogenic facsimiles of Salmonella typhimurium that potently activate dendritic cells, prime B and T cell responses, and stimulate protective immunity in vivo. J Immunol 179(11):7692-7701.
11. Schild S, Nelson E J, & Camilli A (2008) Immunization with Vibrio cholerae outer membrane vesicles induces protective immunity in mice. Infect Immun 76(10):4554-4563.
12. Rivera J, et al. (2010) Bacillus anthracis produces membrane-derived vesicles containing biologically active toxins. Proc Natl Acad Sci USA 107(44):19002-19007.
13. Sotolongo F, et al. (2007) Cuban Meningococcal BC Vaccine: Experiences & Contributions from 20 Years of Application. MEDICC Rev 9(1):16-22.
14. Rodriguez L, et al. (2011) Proteoliposomes from Mycobacterium smegmatis induce immune cross-reactivity against Mycobacterium tuberculosis antigens in mice. Vaccine 29(37):6236-6241.
15. Rezwan M, Laneelle M A, Sander P, & Daffe M (2007) Breaking down the wall: fractionation of mycobacteria. Journal of microbiological methods 68(1):32-39.
16. Saha D C, et al. (2007) Serologic evidence for reactivation of cryptococcosis in solid-organ transplant recipients. Clin Vaccine Immunol 14(12):1550-1554.
17. Gehring A J, Dobos K M, Belisle J T, Harding C V, & Boom W H (2004) Mycobacterium tuberculosis LprG (Rv1411c): a novel TLR-2 ligand that inhibits human macrophage class II MHC antigen processing. J Immunol 173(4):2660-2668.
18. Rezwan M, Grau T, Tschumi A, & Sander P (2007) Lipoprotein synthesis in mycobacteria. Microbiology 153(Pt 3):652-658.
19. Flynn J L & Chan J (2001) Immunology of tuberculosis. Annu Rev Immunol 19:93-129.
20. Cole S T, et al. (1998) Deciphering the biology of Mycobacterium tuberculosis from the complete genome sequence. Nature 393(6685):537-544.
21. Layre E, et al. (2011) A Comparative Lipidomics Platform for Chemotaxonomic Analysis of Mycobacterium tuberculosis. Chem Biol 18(12):1537-1549.
22. Sartain M J, Dick D L, Rithner C D, Crick D C, & Belisle J T (2011) Lipidomic analyses of Mycobacterium tuberculosis based on accurate mass measurements and the novel “Mtb LipidDB”. J Lipid Res 52(5):861-872.
23. Fransen F, et al. (2010) The structure of Neisseria meningitidis lipid A determines outcome in experimental meningococcal disease. Infect Immun 78(7):3177-3186.
24. Kunnath-Velayudhan S, et al. (2010) Dynamic antibody responses to the Mycobacterium tuberculosis proteome. Proc Natl Acad Sci USA 107(33):14703-14708.
25. Baena A & Porcelli S A (2009) Evasion and subversion of antigen presentation by Mycobacterium tuberculosis. Tissue Antigens 74(3):189-204.
26. Harding C V & Boom W H (2010) Regulation of antigen presentation by Mycobacterium tuberculosis: a role for Toll-like receptors. Nat Rev Microbiol 8(4):296-307.
27. Lancioni C L, et al. (2011) Mycobacterium tuberculosis lipoproteins directly regulate human memory CD4(+) T cell activation via Toll-like receptors 1 and 2. Infect Immun 79(2):663-673.
28. Parlane N A, et al. (2012) Phosphatidylinositol di-mannoside and derivates modulate the immune response to and efficacy of a tuberculosis protein vaccine against Mycobacterium bovis infection. Vaccine 30(3):580-588.
29. Cooper A M (2009) Cell-mediated immune responses in tuberculosis. Annu Rev

Immunol 27:393-422.
30. Abebe F & Bjune G (2009) The protective role of antibody responses during Mycobacterium tuberculosis infection. Clin Exp Immunol 157(2):235-243.
31. Glatman-Freedman A & Casadevall A (1998) Serum therapy for tuberculosis revisited: reappraisal of the role of antibody-mediated immunity against Mycobacterium tuberculosis. Clin Microbiol Rev 11(3):514-532.
32. Teitelbaum R, et al. (1998) A mAb recognizing a surface antigen of Mycobacterium tuberculosis enhances host survival. Proc Natl Acad Sci USA 95(26):15688-15693.
33. Maglione P J, Xu J, & Chan J (2007) B cells moderate inflammatory progression and enhance bacterial containment upon pulmonary challenge with Mycobacterium tuberculosis. J Immunol 178(11):7222-7234.
34. Palma C, et al. (2008) The LTK63 adjuvant improves protection conferred by Ag85B DNA-protein prime-boosting vaccination against Mycobacterium tuberculosis infection by dampening IFN-gamma response. Vaccine 26(33):4237-4243.
35. Chang-hong S, et al. (2008) Immune responses and protective efficacy of the gene vaccine expressing Ag85B and ESAT6 fusion protein from Mycobacterium tuberculosis. DNA Cell Biol 27(4):199-207.
36. Teixeira F M, et al. (2006) DNA vaccine using Mycobacterium bovis Ag85B antigen induces partial protection against experimental infection in BALB/c mice. Clin Vaccine Immunol 13(8):930-935.
37. Giri P K, Verma I, & Khuller G K (2006) Enhanced immunoprotective potential of Mycobacterium tuberculosis Ag85 complex protein based vaccine against airway Mycobacterium tuberculosis challenge following intranasal administration. FEMS Immunol Med Microbiol 47(2):233-241.

1. A composition comprising a plurality of isolated mycobacterium membrane vesicles.

2. The composition of claim 1, further comprising an immunological adjuvant.

3. The composition of claim 1, further comprising a pharmacologically acceptable carrier.

4. The composition of claim 1, wherein the isolated mycobacterium membrane vesicles are M. tuberculosis membrane vesicles.

5. The composition of claim 1, wherein the isolated mycobacterium membrane vesicles are M. bovis Bacille Calmette Guerin (BCG) Pasteur membrane vesicles.

6. The composition of claim 1, further comprising a tuberculosis vaccine active agent.

7. The composition of claim 6, wherein the tuberculosis vaccine active agent is a M. bovis BCG Pasteur, or a portion thereof.

8. The composition of claim 1, wherein the isolated mycobacterium membrane vesicles are from Mycobacterium avium, Mycobacterium bovis, Mycobacterium diphtheria, Mycobacterium intracellulare, Mycobacterium leprae, Mycobacterium lepraemurium, Mycobacterium phlei, Mycobacterium smegmatis, Mycobacterium fortuitum, Mycobacterium lufu, Mycobacterium paratuberculosis, Mycobacterium habana, Mycobacterium scrofulacium, or Mycobacterium kansasii.

9. The composition of claim 1, wherein the mycobacterium is an H37Rv strain M. tuberculosis.

10. A method of eliciting an immune response in a subject comprising administering to the subject the composition of claim 1 in an amount effective to elicit an immune response.

11. A method of improving the efficacy of an anti-mycobacterial immunization administered to a subject, comprising administering to the subject the composition of claim 1 in an amount effective to improve efficacy of an anti-mycobacterial immunization administered to a subject.

12. The method of claim 11, wherein the anti-mycobacterial immunization is an anti-tuberculosis immunization.

13. The method of claim 11, wherein the tuberculosis vaccine active agent is a M. tuberculosis BCG Pasteur, or a portion thereof.

14. The method of claim 11, wherein the composition is administered to the subject via the same route as the anti-mycobacterial immunization is administered.

15. A method of immunizing a subject against tuberculosis comprising administering to the subject an amount of the composition of claim 4 effective to immunize a subject against tuberculosis.

16. The method of claim 10, wherein the subject is immunocompromised.

17. A composition comprising a plurality of isolated bacterium membrane vesicles isolated from a pathogenic bacterium that produces membrane vesicles.

18-20. (canceled)

21. A method of preparing an isolated mycobacterium membrane vesicles preparation comprising centrifuging a culture of mycobacterium and treating the resultant product so as to remove live mycobacteria and recovering the vesicles, thereby preparing the isolated mycobacterium membrane vesicles preparation.

22. The method of claim 21, wherein the isolated mycobacterium membrane vesicles are M. tuberculosis membrane vesicles.

23. The method of claim 21, wherein the isolated mycobacterium membrane vesicles are M. bovis Bacille Calmette Guerin (BCG) Pasteur membrane vesicles.

24-25. (canceled)
